(19) United States
(12) Patent Application Publication
(10) Pub. No.: US 2026/0240198 A1
(43) Pub. Date: Aug. 20, 2026

(54) POLYMER/MILK-PROTEIN HYDROGELS, CELL SCAFFOLDS AND CULTURED MEAT PRODUCTS MADE THEREWITH, AND ASSOCIATED METHODS

(71) Applicant: The University of Vermont and State Agricultural College, Burlington, VT (US)

(72) Inventors: Rachael Floreani, Burlington, VT (US); Patrick Charron, Burlington, VT (US)

(21) Appl. No.: 18/858,910

(22) PCT Filed: Apr. 22, 2023
(86) PCT No.: PCT/US2023/019509

§ 371 Date: Oct. 22, 2024

Related U.S. Application Data
(60) Provisional application No. 63/333,819, filed on Apr. 22, 2022.

(51) Int. Cl. A23J 3/08; A23J 3/22; B33Y 10/00; C12N 5/077

(52) U.S. Cl. CPC A23J 3/08; A23J 3/227; B33Y 10/00; C12N 5/0653; C12N 5/0658; C12N 2510/00; C12N 2531/00; C12N 2533/50; C12N 2537/10

(57) ABSTRACT

Hydrogels that comprise one or more polymers and one or more milk proteins, wherein the polymer(s) and the milk protein(s) form an interconnected polymeric network via conjugation. In some embodiments, molecules of the polymer(s) are chemically modified to promote conjugation within the hydrogel. In some embodiments, molecules of the milk protein(s) are chemically modified to promote conjugation within the hydrogel. In some embodiments, molecules of both the polymer(s) and the milk protein(s) are chemically modified to promote conjugation(s) within the hydrogel to create the interconnected polymeric network. Examples of conjugation include, but are not limited to physical chain entanglement, ionic bonding, covalent bonding, secondary bonding, and hydrophobic-hydrophobic interactions, among others. The hydrogels can be enhanced with one or more cell-adhesion agents. The hydrogels can be used to form microcarriers and/or cell scaffolds for holding and proliferating living cells for creating comestible products, such as cultivated meat products, among others.

### CROSS-REFERENCE TO RELATED APPLICATIONS

This application claims the benefit of priority of U.S. Provisional Patent Application Ser. No. 63/333,819, filed Apr. 22, 2022, and titled “Milk-Protein-Conjugated Biopolymers For In Vitro Meat Culture”, the entire contents of which are incorporated by reference herein for all purposes.

### FIELD

The present disclosure generally relates to the field of cultured meat products. In particular, the present disclosure is directed to polymer/milk-protein hydrogels, cell scaffolds and cultured meat products made therewith, and associated methods.

### BACKGROUND

Hydrogels have many applications. For example, hydrogels are used in the medical field to create cell scaffolds that support human cell growth for creating artificial tissue and in the food field to create cell scaffolds for cultivating animal cells to create cultivated meat products. Both of these fields continue to expand as parts of society move away from using traditional meat production (via natural meats and tissue) for ethical and sustainability reasons, among others. Consequently, improvements in the technology underlying these applications are highly desirable, especially improvements that decrease cost, use biocompatible materials, simplify production, use commonly available materials, use industrial (e.g., dairy industry) waste, and/or improve the end product, among other things.

### SUMMARY

In one implementation, the present disclosure is directed to a hydrogel, which includes polymer molecules of at least one polymer, and protein molecules of at least one milk protein, wherein the polymer molecules or the protein molecules or both the polymer molecules and the protein molecules are chemically modified so that the polymer molecules and the protein molecules form an interconnected network.

In another implementation, the present disclosure is directed to a cell scaffold for culturing biological cells. The cell scaffold includes the hydrogel described in the paragraph immediately above, wherein the hydrogel is tuned for culturing the biological cells.

In yet another implementation, the present disclosure is directed to a cultivated comestible product, which includes the cell scaffold described in the paragraph immediately above, wherein the cell scaffold comprises an interconnected network of the polymer molecules and the protein molecules, and biological cells seeded into the interconnected network.

In another implementation, the present disclosure is directed to a method of making a hydrogel. The method includes providing milk-protein molecules, providing polymer molecules, and causing the milk protein and polymer molecules to conjugate with one another so as to form an interconnecting network.

In still another implementation, the present disclosure is directed to a method of making a three-dimensional (3D) cell scaffold designed and configured to receive a plurality of biological cells. The method includes providing the hydrogel made in accordance with the method described immediately above and forming the hydrogel into the 3D cell scaffold.

In yet another implementation, the present disclosure is directed to a method of making a cultivated comestible product. The method includes providing the 3D cell scaffold made using the method described above, wherein the 3D cell scaffold includes an interconnecting network of the polymer molecules and the milk-protein molecules, and seeding the interconnecting network with biological cells.

### BRIEF DESCRIPTION OF THE DRAWINGS

For the purpose of illustration, the accompanying drawings show aspects of one or more embodiments of the invention(s). However, it should be understood that the invention(s) of this disclosure is/are not limited to the precise arrangements and instrumentalities shown in the drawings, wherein:

FIG. 1 is a representation of an alginate molecule in the presence of a divalent cation that can provide ionic bonding between the alginate molecule and other molecules within a hydrogel of the present disclosure;

FIG. 2A is a representation of the alginate molecule of FIG. 1 that has been chemically modified via methacrylation to include acrylate groups to provide covalent crosslinking through controlled radical polymerization;

FIG. 2B is a graph of each of storage modulus (G′) and loss modulus (G″) versus time for differing wavelengths of photonic stimulation for a hydrogel precursor containing the methacrylated alginate represented in FIG. 2A;

FIG. 3 is a representation of the chemically modified alginate molecule of FIG. 2A that has been further chemically modified to add aldehyde groups that provide functionality to allow additional crosslinking to primary amine groups of milk protein(s) within a hydrogel of the present disclosure;

FIG. 4A is a representation of the chemically modified alginate molecule of FIG. 3 in the presence of a hydrophobic molecule and that has been further chemically modified to add cyclodextrin for creating hydrophobic-hydrophobic conjugation with other hydrophobic molecules;

FIG. 4B is a representation of multiple alginate molecules of FIG. 4A conjugated with one another via hydrophobic-hydrophobic interactions among added cyclodextrin and hydrophobic molecules;

FIG. 4C is a representation of an interconnected polymeric network of an example hydrogel of the present disclosure, illustrating conjugations and/or crosslinking between differing polymer chains and conjugations and/or crosslinked between similar polymer chains;

FIG. 5 illustrates a process for chemically modifying alginate molecules to link cyclodextrin molecules to the alginate molecules via (CH₂)₆ and (CH₂)₂ carbon chains;

FIG. 6A is a representation of an alginate molecule chemically modified at the carboxyl site;

FIG. 6B is a representation of an alginate molecule chemically modified at the hydroxyl site, carboxyl site, and polymer backbone;

FIG. 6C is a representation of an alginate molecule chemically modified at the hydroxyl site;

FIG. 6D is a representation of an alginate molecule chemically modified at the hydroxyl site and polymer backbone;

FIG. 6E is a representation of an alginate molecule chemically modified at the polymer backbone;

FIG. 6F is a representation of an alginate molecule chemically modified at the hydroxyl and carboxyl sites;

FIG. 7A is a representation of the chemically modified alginate molecule of FIG. 3 further chemically modified to include a cell-adhesion-promoting (CAP) functional group, here, an arginylglycylaspartic acid (RGD) functional group;

FIG. 7B is a representation of the chemically modified alginate molecule of FIG. 4A further chemically modified to includes a (CAP) functional group, here, an RGD functional group;

FIG. 8 is a graph of transmittance versus wavelength for each of a native whey protein isolate (WPI) and a methacrylated WPI;

FIG. 9A is a graph of viscosity versus shear rate for the native WPI and the methacrylated WPI of FIG. 8;

FIG. 9B is graph of shear modulus versus time for the native WPI and the methacrylated WPI of FIG. 8;

FIG. 10 is a graph of swell ratio and weight loss for each of a methacrylated alginate (Alg-MA)/WPI hydrogel and an Alg-MA/methacrylated WPI (WPI-MA) hydrogel, as well as an Alg-MA control for comparison;

FIG. 11 is a graph of unconfined compressive modulus for each of an Alg-MA/WPI hydrogel and an Alg-MA/WPI-MA hydrogel, as well as an Alg-MA control for comparison; and FIG. 12 is an array of images of live cells performed after 24 hours of muscle cell proliferation in each of an Alg-MA/WPI hydrogel of the present disclosure and Alg-MA/WPI-MA hydrogel of the present disclosure, as well as an Alg-MA control for comparison.

### DETAILED DESCRIPTION

### General Description

Unless otherwise defined, all terms of art, notations and other scientific terminology used herein are intended to have the meanings commonly understood by those of skill in the art. In some cases, terms with commonly understood meanings are defined herein for clarity and/or for ready reference, and the inclusion of such definitions herein should not necessarily be construed to represent a difference over what is generally understood in the art.

As used herein, the singular forms “a,” “an,” and “the” include the plural referents unless the context clearly indicates otherwise. The terms “include,” “such as,” and the like are intended to convey inclusion without limitation, unless otherwise specifically indicated.

As used herein, the term “comprising” also specifically includes embodiments “consisting of” and “consisting essentially of” the recited elements, unless specifically indicated otherwise.

As used herein, the term “about” when used with a corresponding numeric value refers to ±20% of the numeric value, typically ±10% of the numeric value, often ±5% of the numeric value, and most often ±2% of the numeric value. In some embodiments, the term “about” can mean the numeric value itself. In certain embodiments, where applicable, the term “about” indicates the designated value(s)±one standard deviation of that/those value(s).

As used herein, the term “hydrogel” would be known to one of ordinary skill in the art and includes crosslinked polymeric material that has three-dimensional polymer networks. Hydrogels can be prepared from natural, synthetic or synthetic/natural hybrid polymers. Polysaccharide hydrogels can be formed by covalent crosslinking, chemical conjugation, esterification and polymerization. Methods for synthesizing hydrogels are known to those of ordinary skill in the art and include different polymerization methods using both chemical and physical crosslinking routes. For example, chemically crosslinked hydrogels can be synthesized using methods including, but not limited to, chain growth polymerization, addition and condensation polymerization and gamma and electron beam polymerization. Additionally, physically crosslinked hydrogels can be synthesized using methods included, but not limited to, ionic interaction, crystallization, stereocomplex formation, hydrophobized polysaccharides, protein interaction and hydrogen bond.

In some aspects, the present disclosure is directed to hydrogels comprising molecules of at least one polymer and molecules of at least one milk protein in which the molecules of the polymer(s) and/or the molecules of the milk protein(s) have been chemically modified with at least one chemical modification that allow the molecules to conjugate with one another so as to form an interconnecting polymer network within the hydrogel. In some embodiments, such a hydrogel of the present disclosure can be used for cultivating one or more types of biological cells, including myocytes, adipocytes, hepatocytes, and lipocytes, of one or more living creatures, such as, but not limited to, mammals, reptiles, amphibians, and fish, among others. In some embodiments, the hydrogel can be used to cultivate stem cells (e.g., pluripotent or multipotent stem cells, induced pluripotent stem cells, etc.) or immortalized cells. Additional examples of cells contemplated in the present disclosure and methods for immortalizing cell lines for cultivated meat are provided in U.S. Patent Publication No. US 2020/0140821 A1 titled “EX VIVO MEAT PRODUCTION”, published May 7, 2020, in the names of Elfenbein et al., the disclosure of which is incorporated by reference herein. In some embodiments, end products made using a hydrogel of the present disclosure can include comestibles, such as cultivated meat products, including meat products that closely resemble and/or mimic natural meat products in terms of shape (e.g., a natural beef ribeye steak, a natural salmon fillet, etc.) and/or composition (e.g., fat marbling, layering/segmentation, etc.). In this connection, in some embodiments the hydrogels can provide or be processed into three-dimensional (3D) cell scaffolds that each support the adhesion of seed cells, promote the growth of the seed cells, and/or provide enhanced nutritional value when the end product is a comestible. Those skilled in the art will readily appreciate the wide scope of cultivated comestibles that can be creating using a hydrogel of the present disclosure and/or made in accordance with a methodology of the present disclosure.

### Polymer(s)

A polymer of a hydrogel of the present disclosure may be any polymer that is suitable for forming the desired interconnecting polymer network of the hydrogel and is compatible with the application at issue. Regarding the latter, for example, for a hydrogel used to create a comestible, each polymer may need to be a food-grade polymer, for example, as approved by an appropriate authority, such as the Food and Drug Administration in the U.S. In some embodiments, a polymer may be a biopolymer such as, but not limited to, a plant-based biopolymer, such as, but not limited to, starches, celluloses, pectins, glycogens, and polysaccharides. In some embodiments, a polysaccharide may be a naturally occurring polysaccharide, such as, for example, hyaluronan, dextran, chitosan, chondroitin, alginate, or agarose, among others. In some embodiments, a plant-based polysaccharide may be an alginate. It is noted that as referred to herein and in the appended claims, any biopolymer noted can be in any effective state, such as a native state or a modified state, for example, a genetically modified state or a denatured state, among others, unless specifically indicated otherwise.

A polymer of a hydrogel of the present disclosure may be homopolymer, a heteropolymer, or a polymer blend, such as a homopolymer-copolymer blend, among others. When multiple polymers are present in a hydrogel of the present disclosure, only one, fewer than all, or all of the polymers may be chemically modified in one or more differing ways for producing the conjugation(s) desired interconnecting the polymer network of the hydrogel. In some embodiments, the polymer(s), for example any one or more of the polymers mentioned above or other suitable polymer(s), may be present in an amount, either in a weight percentage or a volume percentage of the hydrogel (hydrated or dehydrated), in an amount of about 0.001% to about 99.999%, of about 0.01% to about 80%, of about 0.01% to about 50%, of about 0.1% to about 25%, among other ranges.

### Milk Protein(s)

A milk protein of a hydrogel of the present disclosure refers to proteins or protein equivalents and/or variants that can be obtained from the natural milk of any suitable animal, such as a dairy cow, a goat, a sheep, a buffalo, a camel, or a yak, among others. Natural milk includes water, lactose, proteins, lipids, and minerals, among other things, including pigments, enzymes, and trace amounts of gases. It is noted that while a hydrogel of the present disclosure includes one or more milk proteins, in some embodiments the hydrogel may include one or more, or none, of the other components of the milk as may be desired for a particular hydrogel composition. For a hydrogel of the present disclosure intended for cultivating living cells, the milk protein(s) may be selected based on its/their cell-adhesion characteristic(s) and/or cell-proliferation characteristic(s). In some embodiments, milk protein(s) can replace an expensive cell adhesion ligand, specifically, an arginylglycylaspartic acid (RGD) peptide motif, and/or other animal components such as collagen and fibronectin. In addition and as discussed below, a milk protein of the present disclosure may be modified to enhance its cell-adhesion ability.

Milk proteins generally fall into one or the other of two families, namely, casein and whey. The casein family proteins generally consist of several types (αs1, αs2, β and κ), with a mix of α-s1 and α-s2 (i.e., α-casein) predominating. The whey family proteins generally consist of β-lactoglobulin, α-lactalbumin, blood serum albumin, immunoglobulins, lactoferrin, transferrin, and a variety of minor proteins and enzymes, with β-lactoglobulin predominating. In some embodiments of a hydrogel of the present disclosure, whey protein(s) can be preferred. In some embodiments of a hydrogel of the present disclosure, β-lactoglobulin can be preferred because of its naturally good cell-adhesion properties. When whey protein is used in a hydrogel of the present disclosure, the whey protein may be provided in any suitable form, such as, for example, part of a whey protein isolate (WPI) or part of a whey-protein concentrate, among others. In some embodiments, using whey protein may be desirable when they are a residual waste product of other processes and thus allow for a sustainable source and means to reduce current industrial waste.

As with a polymer used in creating a hydrogel of the present disclosure, it is noted that as referred to herein and in the appended claims, any milk protein noted can be in any effective state, such as a natural state or a modified state, such as a genetically modified state (e.g., recombinant) or a denatured state, among others, unless specifically indicated otherwise. When multiple milk proteins are present in a hydrogel of the present disclosure, the multiple proteins may be of differing types from the same animal (e.g., multiple whey proteins, whey protein(s) and casein), may be of the same type(s) from differing animals, or both of differing types from the milk of the same animal and of the same type(s) from differing animals. Fundamentally, there are no limitations on the milk protein(s) that can be used in creating a hydrogel of the present disclosure. In some embodiments, the milk protein(s), for example any one or more of the milk proteins mentioned above, may be present in an amount, either in a weight percentage or a volume percentage of the hydrogel (hydrated or dehydrated), in an amount of about 0.001% to about 99.999%, of about 0.001% to about 80%, of about 5% to about 50%, of about 10% to about 30%, among other ranges.

### Chemical Modification(s)

Chemical modifications pertinent to the formation of a hydrogel of the present disclosure include 1) chemical modifications that can be used singly or in various combinations with one another to cause or promote formation of an interconnecting polymer network within a hydrogel of the present disclosure and 2) chemical modifications that can be used to cause or promote the adhesion of living cells to the interconnecting polymer network. Examples of each of these chemical modifications are discussed below.

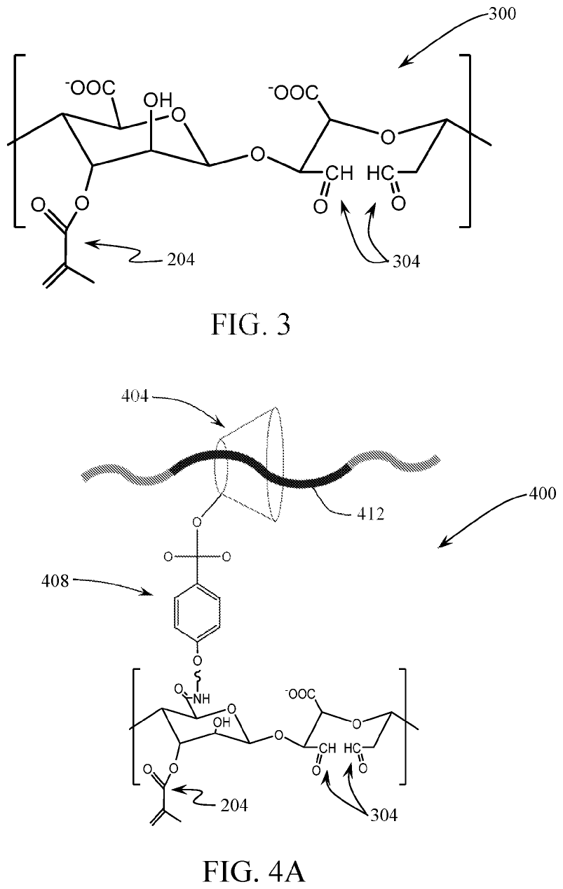

Chemical Modifications for Forming Polymer Network. Each polymer and/or each milk protein may be chemically modified using one or more chemistries to create or promote conjugation(s) of the molecules within a hydrogel of the present disclosure so as to form an interconnected polymer network that comprises the molecules, for example, to form an extracellular matrix (ECM) for cultivating living cells. Depending on the type(s) of chemical modification(s), the conjugation(s) may be of any suitable type, such as ionic crosslinking, covalent crosslinking through controlled radical polymerization (e.g., of the polymer(s)), reversible covalent crosslinks (e.g., reactions of primary amines within the milk protein(s)), and hydrophobic-hydrophobic interactions (e.g., via cyclodextrin bonding onto one or more types of polymers, (e.g., polysaccharides) among others. Examples of these chemical modifications are described below and illustrated in the accompanying FIGS. 2A through 6B in the context of the polymer being alginate. Those skilled in the art will readily appreciate that while these examples involve alginate, the same or similar chemistries can be used for other biopolymers and polymers. As mentioned above, each of these chemical modifications can be used singly, in combination with one another, or in any subcombination with one another.

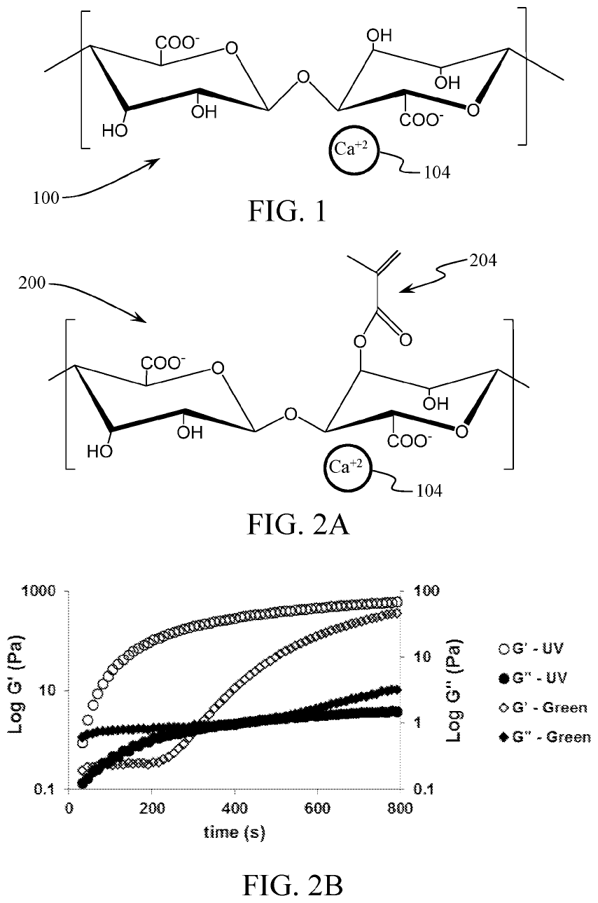

FIG. 1 shows an example of a “backbone” alginate molecule 100 in the presence of a divalent cation 104 (here, a divalent calcium cation) that can create divalent cation-initiated crosslinking with another alginate molecule (not shown). Cations other than calcium cations, including other divalent cations, can be additionally or alternatively used.

FIG. 2A shows an example of a chemically modified backbone alginate molecule 200 chemically modified via an acrylation chemistry to provide the modified alginate molecule with, for example, methacrylate groups 204 (here, via methacrylic anhydride (MA)) to allow covalent crosslinking through controlled radical polymerization, for example, via photonic stimulation. In FIG. 2A, the covalent crosslinking via the methacrylate groups 204 provides a dual-crosslinking with the divalent ionic crosslinking via the cation 104 of FIG. 1. In this example, the divalent cation 104 is also present. However, in other embodiments, the divalent cation 104 is not present. FIG. 2B illustrates the storage modulus and loss modulus versus time for ultraviolet light (UV) and green light induced crosslinking of the methyl acrylate modified alginate molecule 200 of FIG. 2A.

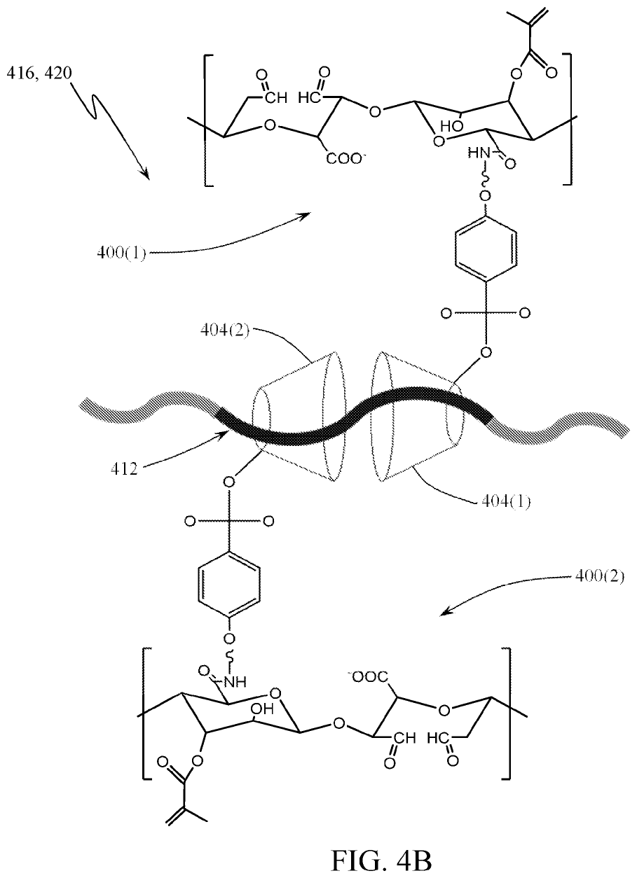

FIG. 3 shows an example of a chemically modified backbone alginate molecule 300 that is similar to the chemically modified alginate molecule 200 of FIG. 2A but is further chemically modified to include, in addition to the acrylate groups 204, aldehyde groups 304 using a suitable chemistry. In an example reaction, sodium periodate is used to oxidize the alginate uronic ring, exposing the two aldehydes. Other reactions may be used to expose the aldehydes. In this example, the aldehyde groups 304 provide covalent crosslinking to primary amines (not shown) of the milk protein(s) (not shown) in an overall interconnected polymer network, for example, an ECM. In other embodiments, this same chemistry can be used without the methacrylation chemistry for forming a crosslinked hydrogel comprising alginate dialdehyde and milk protein (not shown).

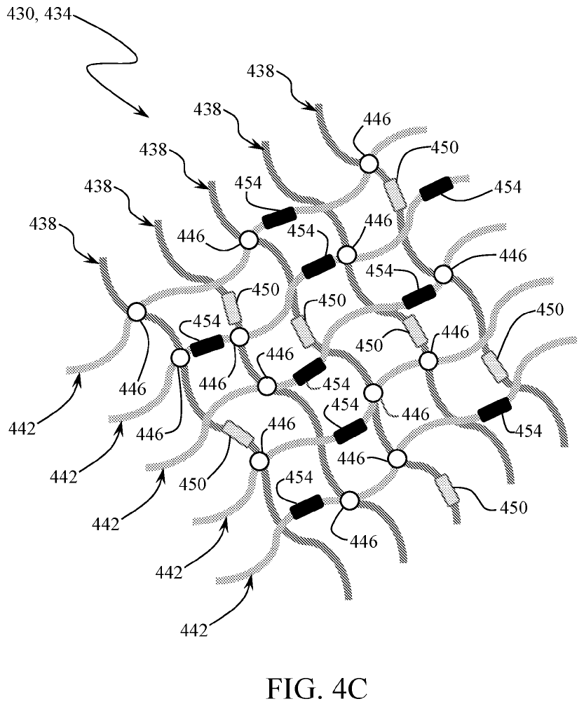

FIG. 4A shows an example of a chemically modified alginate molecule 400 that is similar to the chemically modified alginate molecule 300 of FIG. 3 that includes the acrylate groups 204 and the aldehyde groups 304 but is further chemically modified to include cyclodextrin (CD) 404 using a suitable chemistry. In this example, the CD 404 is linked to the backbone of the modified alginate molecule via polyethylene glycol (PEG) 408. In some embodiments, the CD 404 can serve a dual role. It can entrap milk protein 412 via hydrophobic-hydrophobic interactions as shown, or in the presence of a tri-block copolymer comprising two hydrophilic and a hydrophobic region it can form a self-healing hydrogel based on similar hydrophobic-hydrophobic interaction. In this example, the milk protein 412 forms a physical bond with the CD 404 such that pairs (not shown, but see FIG. 4B) of alginate molecules 404 that each have the CD 404 modification can conjugate with one another via the hydrophobic-hydrophobic interactions with one or more suitable hydrophobic molecules, such as the milk protein of this example. Those skilled in the art will readily appreciate that molecules other than CD having hydrophobic affinity can be used, as well as that linking agents other than PEG can be used in place of or in addition to the PEG. The example alginate molecule 400 of FIG. 4A is light-responsive, capable of multiple crosslinking methods, and self-healing, and can form reversible physical and covalent interaction.

FIG. 4B illustrates an example of a conjugation of a plurality of chemically modified alginate molecules, here, two chemically modified alginate molecules 400(1) and 400(2) each being the same as the chemically modified alginate molecule 400 of FIG. 4A. In this example, the chemically modified alginate molecules 400(1) and 400(2) are conjugated with one another via their respective CD 404(1) and 404(2) via the hydrophobic-hydrophobic interactions of the CD with the milk protein 412. As can be appreciated, this conjugation partially forms an interconnected polymeric network 416 of a CD-modified alginate (Alg-CD)/milk protein hydrogel 420 of the present disclosure.

Expanding on the example interconnecting network 416 of the hydrogel of FIG. 4B, FIG. 4C illustrates a broader example of an interconnected polymeric network 430 of an example hydrogel 434 of the present disclosure. In the example of FIG. 4C, the interconnected polymeric network 430 comprises first polymer chains 438 and second polymer chains 442 that are conjugated with one another via heteroconjugations 446. Within individual ones of the first polymer chains 438 and individual ones of the second polymer chains 442, the individual polymers (not individually labeled to avoid cluttering the figure) are conjugated with one another, respectively, via homoconjugations 450 and 454. As will be appreciated from reading this entire disclosure, each of the heteroconjugations 446 and each of the homoconjugations 450 and 454 may be any suitable type of conjugation, including, but not limited to, ionic crosslinking, covalent crosslinking through controlled radical polymerization, secondary crosslinking, and hydrophobic-hydrophobic interactions, among others.

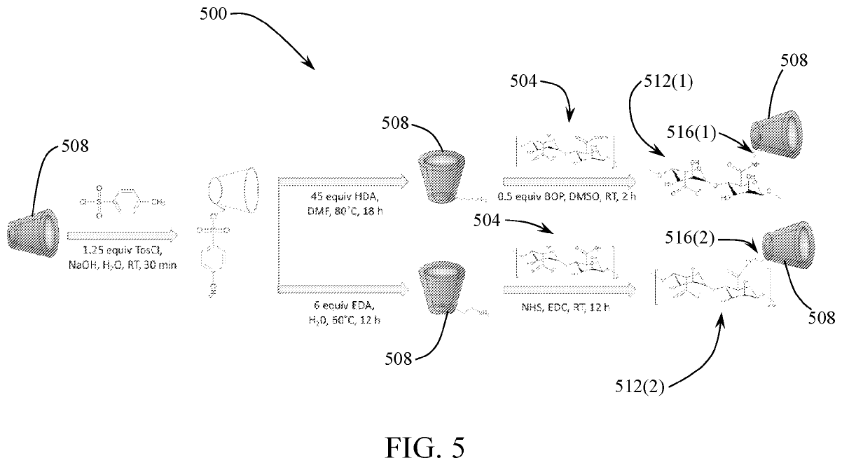

FIG. 5 illustrates an example process 500 functionalizing alginate 504 with CD 508 to create two chemically modified alginates 512(1) and 512(2) having hydrocarbon chains 516(1) and 516(2), respectively, of differing lengths. In this example, the hydrocarbon chain 516(1) is (CH₂)₆ and the hydrocarbon chain 516(2) is (CH₂)₂. In FIG. 5, the following apply: “Alg” is alginate; “C6” is (CH₂)₆; “C2” is (CH₂)₂; “TosCl” is 4-toluenesulfonyl chloride; “NaOH” is sodium hydroxide; “RT” is room temperature; “min” is minute(s); “equiv” is equivalent(s); “HDA” is 1,6-hexanediamine; “DMF” is dimethylformamide; “EDA” is ethylene diamine; “h” is hour(s); “BOP” is (benzotriazol-1-yloxy)tris(dimethylamino) phosphonium hexafluorophosphate; “DMSO” is dimethyl sulfoxide; “NHS” is N-hydroxysuccinimide; and “EDC” is 1-ethyl-3-(3dimethylaminopropyl)carbodiimide. As those skilled in the art will readily appreciate, the process 500 illustrated in FIG. 5 is merely exemplary and non-limiting.

As noted, the above chemical modifications are merely examples of the types of chemical modifications that can be used to chemically modify the polymer(s) and/or the milk protein(s) to create a hydrogel of the present disclosure. A partial list of suitable chemical modifications to create or promote covalent bonding, secondary bonding, and/or hydrophobic-hydrophobic interactions includes, but is not limited to acrylation, methacrylation, oxidation, carbodiimide modification, diamine modification, dihydrazide modification, esterification, acetylation, phosphorylation, sulfation, alkylation, ethylation, arylation, amination, amide modification, pegylation, graft copolymerization, and aldehyde modification (monoaldehyde or polyaldehyde), among others. While alginate is used as the backbone polymer, those skilled in the art will understand that the example chemical modifications can be applied to other polymers, such as other polysaccharides and other biopolymers more generally. A partial list of biopolymers that can be modified using the example chemical modification includes, but is not limited to, polysaccharides (e.g., alginate, agarose, chitosan, chitin, gellan gum, gum arabic, carrageenan, cellulose, carboxymethylcellulose, methyl cellulose, xanthan gum, dextran, dextran sulfate, Hyaluronan, heparin, heparin sulfate, chondroitin sulfate, dermatan sulfate, keratin sulfate, carob gum, pullulan, starches, pectins, glycogens), shorter saccharides (e.g., saccharides, disaccharides, trisaccharide, ogliosaccharides), and polymeric proteins (e.g., fibrin, collagen, fibronectin, laminin, gelatin, elastin), among others. For alginate, in particular and in some embodiments, there are generally three major groups of chemical modifications that can be performed based on the available reactive groups, but these and combinations thereof may change depending on polymer choice. These are hydroxyl modification (including methacrylation), carboxyl modification (including cyclodextrin and RGD chemistries), and modification of the polymer backbone (including oxidation).

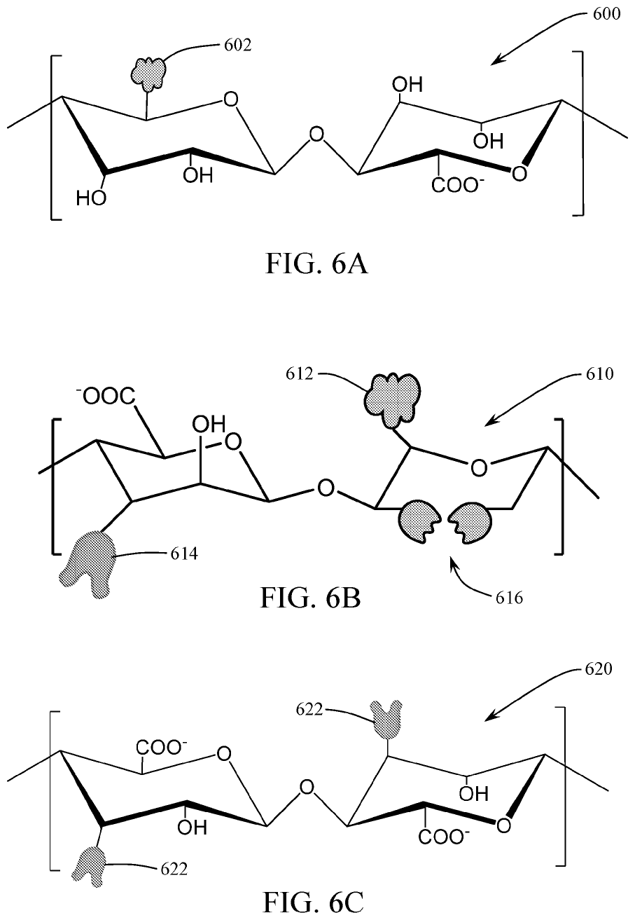

FIGS. 6A through 6F illustrate some example modifications that can be made to a polymer that is used in a hydrogel of the present disclosure. It is noted that all of these examples are based on alginate as the polymer. However, those skilled in the art will readily appreciate that similar modifications can be made to other polymers and that these chemical modifications are merely examples. FIG. 6A shows a chemically modified polymer molecule 600 that has a chemical modification 602, such as a cyclodextrin modification, present at the carboxyl site of the polymer molecule. FIG. 6B shows a chemically modified polymer molecule 610 that has chemical modifications 612, 614, and 616, present, respectively, at the hydroxyl site, carboxyl site, and polymer backbone of the polymer molecule. For example, the chemical modification 612 at the hydroxyl site may be, for example, a methacrylation modification, the chemical modification 614 at the carboxyl site may be, for example, a cyclodextrin modification, and the chemical modification 616 at the polymer backbone may be, for example, a dialdehyde modification. FIG. 6C shows a chemically modified polymer 620 that has a chemical modification 622, such as a methacrylation modification, at the hydroxyl site of the polymer molecule.

FIG. 6D shows a chemically modified polymer molecule 630 that has chemical modifications 632 and 634, present, respectively, at the hydroxyl site and polymer backbone of the polymer molecule. For example, the chemical modification 632 at the hydroxyl site may be, for example, a methacrylation modification and the chemical modification 634 at the polymer backbone may be, for example, a dialdehyde modification. FIG. 6E shows a chemically modified polymer 640 that has a chemical modification 642, such as a dialdehyde modification, at the polymer backbone of the polymer molecule. FIG. 6F shows a chemically modified polymer molecule 650 that has chemical modifications 652 and 654, present, respectively, at the hydroxyl site and carboxyl site of the polymer molecule. For example, the chemical modification 652 at the hydroxyl site may be, for example, a methacrylation modification and the chemical modification 654 at the carboxyl site may be, for example, a cyclodextrin modification.

In addition, while light can be used as the stimulus for polymerization, other types of stimuli can be used with the appropriate modification to the relevant chemistry. For example, other stimuli other than photonic stimulation can include, but not be limited to the addition of chemical compounds, electric current, and/or magnetic fields or changes to temperature, strain, pressure, humidity, and/or pH. For each of these stimuli, each may be controlled in a manner that controls the corresponding polymerization. For example, if polymerization is stimulated at or above a polymerization temperature, Tp, then components for making a hydrogel can be mixed at a temperature lower than Tp and then the temperature of the mixture raised to equal to or greater than Tp when polymerization is desired. In this example, heat may be added in any suitable manner known in the art. As another example, if polymerization is stimulated at or below a polymerization pH, pHp, then components for making a hydrogel can be mixed at a pH higher than pHp and then the pH of the mixture lowered to equal to or less than pHp when polymerization is desired. For polymerization occurring as a function of pH, a pH adjuster can be added to the mixture at a desired/necessary time to adjust the pH of the mixture accordingly.

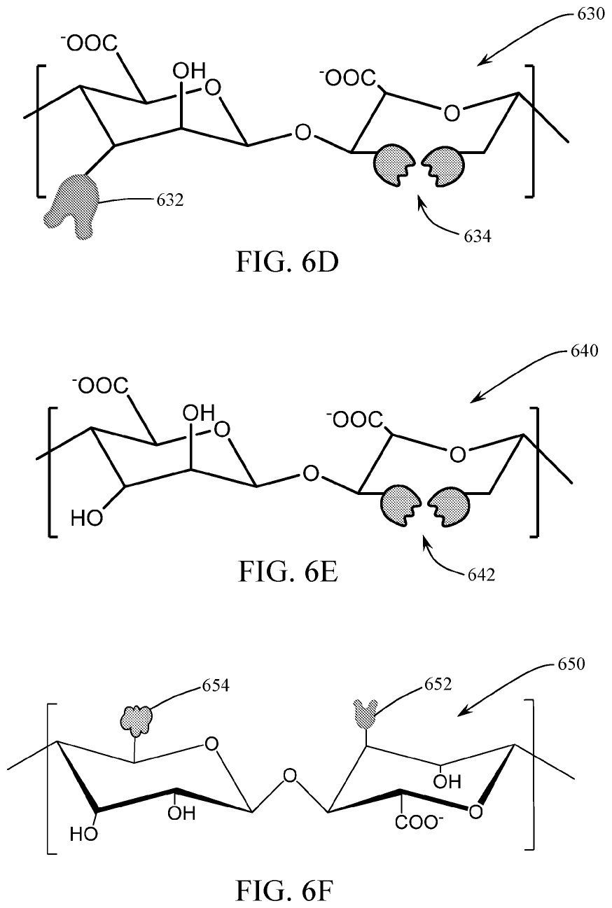

Chemical Modifications for Promoting Cell Adhesion: In embodiments of hydrogels made in accordance with the present disclosure made to support live-cell cultivation, the extra-cellular matrix, or cell scaffold, may be chemically modified to promote adhesion of the living cells to the scaffold. For example, molecules of the milk protein(s) and/or molecules of the polymer(s) can be chemically modified by adding one or more cell-adhesion-promoting (CAP) functional groups. In some embodiments, such functional group(s) can be covalently bonded to molecules of the milk protein(s) and/or to molecules of the polymer(s) using any suitable type of bond formation. In the context of covalent bonding of CAP functional groups, crosslinking may be performed via, for example, carbodiimide crosslinker chemistry, disulfide bond formation, or esterification, among others. Examples of CAP molecules that can be used to form the CAP functional groups that attach to the cell scaffold include, but are not limited to arginylglycylaspartic acid (RGD) molecules, collagen molecules, fibronectin molecules, and laminin molecules, among others. FIGS. 7A and 7B show, respectively, the chemically modified alginate molecules 300 and 400 of FIGS. 3 and 4A each further chemically modified to include a CAP functional group, here, an RGD functional group 700, so as to create corresponding functionalized chemically modified alginate molecules 704 and 708. Those skilled in the art will understand that CAP functional groups can be added to hydrogels of the present disclosure using conventional methods adopted to the relevant chemistry(ies) of the hydrogel using only knowledge known in the art.

### Cell Scaffolds (ECMs)

As noted above, a hydrogel of the present disclosure can be used to provide or make cell scaffolds, also referred to herein as ECMs, for growing and proliferating one or more types of living cells, such as to make cultured meat, to repair or replace human tissue, or to create tissue samples for testing, among other things. Using one or more of the above-identified chemical modifications and/or any other suitable modifications, properties of a cell scaffold of the present disclosure can be tuned for specific applications by altering components through additional chemical modification and/or altering the process of forming the hydrogel, for example by selecting one or more chemistries compatible with the application of the cell scaffold. Such modifiable properties include degradation, mechanical properties, swelling, and porosity, among others. Altering or tuning the properties of the material can allow for a more diverse range of cell types, including, as noted above, cells of differing meats, e.g., beef, poultry, fish, pork, etc., and differing tissue types, e.g., muscle and fat. Tuning these properties can allow more accurate imitation of complex matrices found in nature. Those skilled in the art will be able to select materials and chemistries suitable for making a hydrogel suitable for a cell scaffold of the present disclosure using knowledge common in the art and using this disclosure as a guide. Similarly, those skilled in the art will be able to tune the parameter(s) of the relevant chemistry(ies) and other processes of forming a cell scaffold of the present disclosure without undue experimentation to arrive at a cell scaffold suitable for the particular application at issue.

A cell scaffold of the present disclosure comprises a 3D structure composed of a hydrogel made in accordance with the present disclosure, such as described above and provided in examples below, and having voids or space to receive or otherwise contain seed living cells and to provide space for the seed cells to proliferate and grow so as to create the desired end product, such as cultivated meat, among other things. A cell scaffold of the present disclosure can be made in any of a variety of ways, with some examples as follows. In one example, a hydrogel can be formed in a mold having the shape of the desired final product. For example, a hydrogel precursor can be placed into a mold, with or without seed cells, and then polymerization can be initiated to create the 3D structure. The 3D structure and the mold can then be separated from one another. In some embodiments, the resulting 3D structure can be dried before seeding with living cells. In a generally related embodiment, a large mass of the hydrogel can be formed, perhaps in a mold, and then one or more 3D shapes can be cut from the large mass to create the 3D structures having the desired shape(s).

In another example, voids within the hydrogel and/or cell scaffold can be created and/or enhanced using a porogen. For example, a porogen can be added to a hydrogel precursor and the hydrogel precursor polymerized to form the hydrogel, i.e., the interconnecting polymer network. The porogen can then be removed from the hydrogel to leave the voids created and/or enhanced by the porogen. In a further example, a mass of hydrogel, hydrogel precursor, or partially crosslinked precursor can be dried (e.g., by lyophilization) and ground-up to create particles that may then be used to create a cell scaffold of a desired shape. In yet another example, the hydrogel may be formed into particles, such as spheres, either before, after full polymerization, or after partial polymerization. The particles may optionally be dried before using, and may be seeded with living cells before, during, or after formation. Each particle may be considered a cell scaffold in and of itself and fully support cell proliferation and growth. In some embodiments, a plurality of such cell-scaffold particles can be aggregated with one another, for example, within a mold, to create a larger cell scaffold having the desired 3D shape. In some embodiments, the aggregation of cell-scaffold particles may be polymerized within the mold and/or may be kept in the mold during the cell proliferation and growth process at least until the cell-scaffold particles and cells form a unitary mass able to be separated from the mold. In still further examples of forming a cell scaffold, the hydrogel, or precursor thereto (e.g., not-yet polymerized mixture) can be electro-sprayed, electrospun, applied as an emulsion, or printed using a 3D printer, layer-by-layer deposition, any additive manufacturing, among others.

### Cultivated Meat Products

As mentioned above, a cell scaffold of the present disclosure, such as any of the cell scaffolds described in the immediately preceding subsection, any cell scaffold apparent to someone of ordinary skill in the art from that description, and any cell scaffold made using a hydrogel made in accordance with the present disclosure, can be used to create any of a variety of cultivated meat products, such as, for example, beef steaks, organ meat (e.g., calf liver, chicken liver, etc.), ground beef, fish fillets, lamb meat, ground chicken, chicken thigh meat, and turkey breast meat, to name just a few, and any hybrid containing meats of two or more differing species of animal.

To create a cultivated meat product of the present disclosure, a cell scaffold and/or a precursor thereto (e.g., an unformed hydrogel, a hydrogel precursor (e.g., not-yet-polymerized mixture or component(s) thereof, etc.)) is seeded with one or more types of living cells, such as myocytes, adipocytes, and lipocytes, depending on the nature and composition of the desired cultivated meat product. For example, if fat marbling is desired in a cultivated ribeye beef steak, bovine adipocytes may be seeded into regions within a cell scaffold wherein the marbled fat is desired, while bovine myocytes are seeded into other regions wherein meat is desired.

In some embodiments and in the context of a hydrogel precursor mixture (e.g., pre-polymerization or after partial conjugation (e.g., ionic crosslinking only)), the living cells may be seeded at a density of about 10⁵ to about 10⁹ cells per milliliter of the hydrogel precursor mixture, of about 10⁵ to about 10⁸ cells per milliliter of the hydrogel precursor mixture, or of about 10⁶ to about 10⁷ cells per milliliter of the hydrogel precursor mixture, among others. In some embodiments, and in the context of a crosslinked hydrogel, the living cells may be seeded at a density of about 105 to about 10⁹ cells per cubic centimeter of the hydrogel, of about 10⁵ to about 10⁸ cells per cubic centimeter of the hydrogel, or of about 10⁶ to about 10⁷ cells per cubic centimeter of the hydrogel, among others. Those skilled in the art will readily appreciate that the foregoing seeding density ranges are merely exemplary and that other seeding densities may be needed to provide optimal solutions in particular applications.

### EXAMPLES

Following are examples that illustrate, among other things, differing chemistries that can be used to create differing hydrogels of the present disclosure that have differing compositions and differing conjugations that create the hydrogels. It is noted that neither the hydrogels themselves nor the chemistries of these examples are necessarily optimized for any particular purpose. Rather, they are provided as illustrations of various principles disclosed herein. Those skilled in the art will readily be able to extract fundamentals from these examples to apply alternative chemistries, use one or more differing polymers and/or one or more differing milk proteins, and/or tune the properties of a desired hydrogel using knowledge common in the art and without undue experimentation.

The following are examples of methods and compositions of the present disclosure. It is understood that various other embodiments may be practiced, given the general description provided herein. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### Example 1: A Methacrylic Anhydride (MA) Methacrylated Alginate (Alg-MA)/MA Methacrylated WPI (WPI-MA) Hydrogel

The milk protein molecules used in this example were obtained from commercially available, non-specific whey protein isolates (WPI) containing 86-92% protein by weight. Methacrylic anhydride was used to chemically methacrylate the whey protein isolate to form whey protein isolate methacrylate (WPI-MA). The product was purified via dialysis, lyophilized, and stored dry until use. In a related experiment, casein was also methacrylated in a similar manner as the WPI.

From here, the WPI-MA was covalently chemically attached to another polymer, here, alginate, that was also methacrylated, in the presence of an initiator. The polymerization reaction was photo-induced using a photo-initiator and green light as the stimulation source. In related experiments on the polysaccharide side, hyaluronan, chitosan, gum arabic, and gelatin were also methacrylated in a similar manner as the alginate.

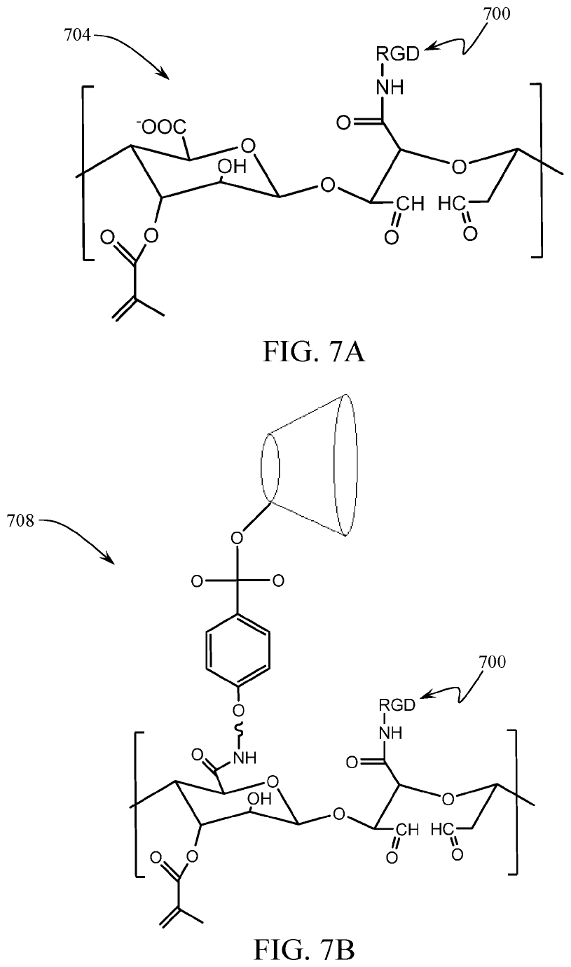

Attenuated total reflectance Fourier transform infrared (ATR-FTIR) spectroscopy was performed on the modified and non-modified proteins to verify the chemical modification of the protein, with the results appearing in FIG. 8. The FTIR results alone were inconclusive, as the peak associated with the methacrylate bonds overlap with peaks present in the non-modified protein. However, the results suggest a chemical modification occurred due to more pronounced peaks present in the WPI-MA sample and slight shifts in the peaks when comparing the WPI and WPI-MA samples. These shifts often occur because of formation of new bonds during the chemical modification. Similar shifts occur in other materials undergoing methacrylation modification.

### Example 2: Preparation of an MA Methacrylated Alginate (Alg-MA)/WPI Hydrogel via Physical Chain Entanglement

Preparation of an Alg-MA/WPI matrix comprising 3% w/v Alg-MA and 10% w/v WPI. This example illustrates an interpenetrating network in which non-modified milk protein is embedded in a crosslinked modified polysaccharide matrix via physical chain entanglement.

Alg-MA was synthesized via an aqueous reaction in the presence of a 20-molar excess of methacrylic anhydride, adjusting the pH to 8.5 with NaOH periodically, followed by purification via dialysis in deionized (DI) water and lyophilization. An Alg-MA/WPI solution was prepared by dissolving Alg-MA and WPI in DI water to final concentrations of 3% w/v and 10% w/v respectively. Alg-MA can be dissolved into a WPI solution, or vice versa. To enable photocrosslinking, a photoinitiator solution capable of releasing free radicals upon exposure to specific light wavelengths was added to the Alg-MA/WPI solution. This example utilized an Eosin Y/1-vinyl-2-pyrrolidone & triethanolamine system (5 μL & 50 μL in 2 mL polymer solution, respectively), which reacts when exposed to green light (525 nm). This hydrogel precursor solution can then be cast into wells or molds and crosslinked to form the final matrix upon exposure to 10 minutes of green light. The resulting hydrogel can be sterilized via UV exposure, among other techniques.

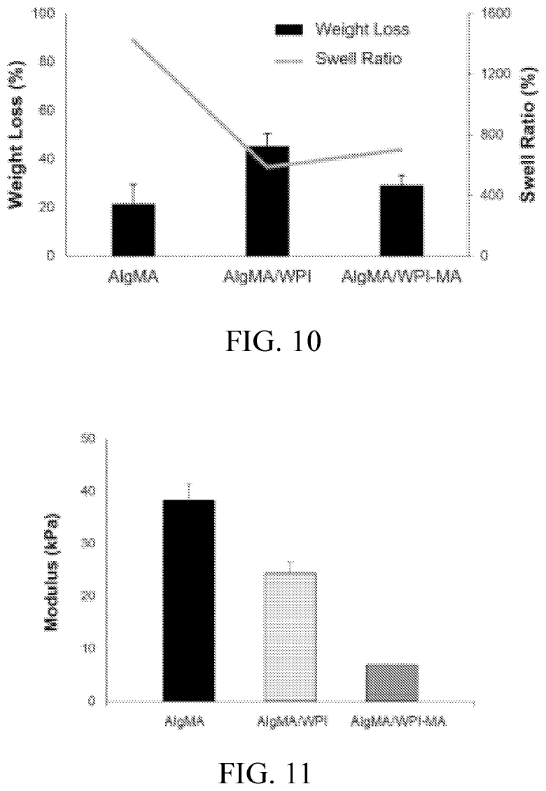

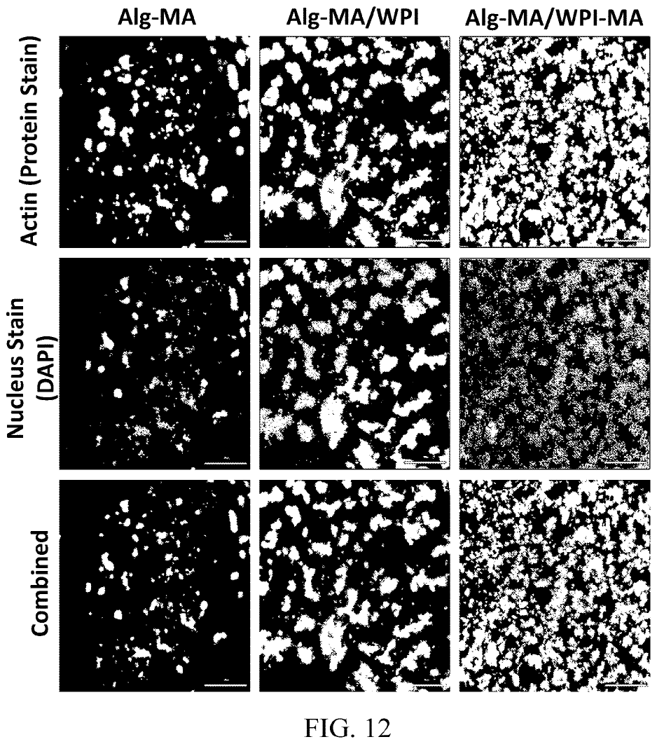

The resultant Alg-MA/WPI hydrogel was characterized to determine the physical properties. Swell ratio and weight loss was determined following a modified ASTM D2765-11 standard with the results appearing in FIG. 10. Unconfined compressive modulus was determined through analysis of the linear region of the stress-strain curves with the results appearing in FIG. 11. Cell proliferation was observed by seeding the hydrogel matrices with C2Cl2 myoblasts with the results appearing in FIG. 12.

### Example 3: Preparation of the Alg-MA/WPI-MA Hydrogel of Example 1 That is Physically Conjugated and Chemically Crosslinked

Preparation of an Alg-MA/WPI-MA matrix comprising 3% w/v Alg-MA and 10% w/v WPI-MA. This example illustrates a matrix formed via covalent bonding between the modified polysaccharide and modified milk protein polymers to form a final structure that is physically and chemically crosslinked.

Alg-MA was synthesized via an aqueous reaction in the presence of a 20-molar excess of methacrylic anhydride, adjusting the pH to 8.5 with NaOH periodically, followed by purification via dialysis in DI water and lyophilization. WPI-MA was synthesized via an aqueous reaction in PBS in the presence of a 24-molar excess of methacrylic anhydride, adjusting the pH with NaOH as needed, followed by purification via dialysis in DI water and lyophilization. An Alg-MA/WPI-MA solution was prepared by dissolving Alg-MA and WPI-MA in DI water to final concentrations of 3% w/v and 10% w/v respectively. Alg-MA can be dissolved into a WPI-MA solution, or vice versa. To enable photocrosslinking, a photoinitiator solution capable of releasing free radicals upon exposure to specific light wavelengths was added to the Alg-MA/WPI-MA solution. This example utilized an Eosin Y/1-vinyl-2-pyrrolidone & triethanolamine system (5 μL & 50 μL in 2 mL polymer solution, respectively), which reacts when exposed to green light (525 nm). This hydrogel precursor solution can then be cast into wells or molds and crosslinked to form the final matrix upon exposure to 10 minutes of green light. The resulting hydrogel can be sterilized via UV exposure, among other techniques.

The resultant Alg-MA/WPI-MA hydrogel was characterized to determine the physical properties. Swell ratio and weight loss was determined following a modified ASTM D2765-11 standard with the results appearing in FIG. 10. Unconfined compressive modulus was determined through analysis of the linear region of the stress-strain curves with the results appearing in FIG. 11. Cell proliferation was observed by seeding the hydrogel matrices with C2Cl2 myoblasts with the results appearing in FIG. 12.

### Example 4: Preparation of a Dialdehyde Modified Alginate (Alg-DA)/WPI Hydrogel That is Physically Entangled and Covalently Crosslinked

Preparation of an Alg-DA/WPI matrix comprising 5% w/v Alg-DA and 10% w/v WPI. This example illustrates a matrix formed via a reaction between aldehyde groups present in Alg-DA to form a final structure that is chemically crosslinked. The primary amines on the WPI protein chains form spontaneous covalent bonds with the aldehydes present on the chemically modified alginate.

Alg-DA with a 10% theoretical degree of oxidation was synthesized via aqueous reaction of alginate in the presence of a 1:10 weight ratio polymer to sodium periodate in a dark environment at room temperature for 24 hours. The reaction was quenched via addition of an equivalent molar ratio of ethylene glycol to sodium periodate, followed by purification via dialysis in DI water and lyophilization. An Alg-DA/WPI solution was made by solubilizing the modified Alg-DA and non-modified WPI in DI water to final concentrations of 5% & 10% respectively. To this solution, an aqueous adipic acid dihydrazide solution was added at a 1:1 molar ratio of adipic acid to aldehyde in the Alg-DA. This solution was then cast into a mold and allowed to sit at room temperature for 45 minutes, after which a hydrogel was observed.

### Example 5: Preparation of an Alg-MA-β-CD/WPI Hydrogel That is Conjugated via Hydrophobic-Hydrophobic Interaction

Preparation of an Alg-MA-β-CD/WPI matrix comprising 5% w/v Alg-β-CD and 10% w/v WPI. This example illustrates a matrix formed via hydrophobic-hydrophobic interactions between the hydrophobic region of the cyclodextrin rings on a modified polysaccharide and hydrophobic regions of the milk proteins.

Alg-MA was synthesized via an aqueous reaction in the presence of a 20-molar excess of methacrylic anhydride, adjusting the pH to 8.5 with NaOH periodically, followed by purification via dialysis in DI water and lyophilization. β-CD (20 g) was dissolved in cold DI water, to which a solution of 4.2 g TosCl in 10 mL acetonitrile was added dropwise and stirred vigorously for 2 hours at room temperature. 2.18 g of solid NaOH dissolved in 10 ml of DI water was added dropwise to adjust the pH to approximately 8.5, followed by 30 minutes of vigorous stirring at room temperature. The solution was refrigerated overnight at 4 C and the resulting β-CD-TosCl precipitate was thoroughly washed five times with ethanol and dried under vacuum. 1.5 g dried β-CD-TosCl was added to 5 mL EDA, stirred under a condenser at 60 C for 24 hours, cooled to room temperature, and precipitated in cold ethanol. The resulting β-CD-EDA precipitate was washed five times in ethanol and dried under vacuum to yield a dry product. 2.7 g Alg-MA was dissolved in 150 0.1 M MES buffer, to which 2 g of EDC and 1.2 g of NHS were added and allowed to stir at room temperature for 30 minutes. 4.5 g-CD-EDA was added and vigorously stirred for 24 hours at room temperature.

The solution was then purified via dialysis and dried via lyophilization to yield dry Alg-MA-β-CD. An Alg-MA-β-CD/WPI solution was prepared by dissolving Alg-MA-β-CD and WPI into DI water to final concentrations of 5% and 10% respectively. To enable photocrosslinking, a photoinitiator solution capable of releasing free radicals upon exposure to specific light wavelengths was added to the Alg-MA-B-CD/WPI-MA solution. This example utilized an Eosin Y/1-vinyl-2-pyrrolidone & triethanolamine system (5 μL and 50 μL in 2 mL polymer solution, respectively), which reacts when exposed to green light (525 nm). The hydrogel precursor solution was cast into a mold, allowed to sit for 10 minutes to allow for hydrophobic-hydrophobic interactions, and crosslinked via exposure to 10 minutes of green light, after which a hydrogel was observed.

### Example 6: Preparation of an Glycidyl Methacrylate (GM) Methacrylated Alginate (Alg-GM)/WPI Hydrogel via Physical Chain Entanglement

Preparation of an Alg-GM/WPI matrix comprising 3% w/v Alg-GM and 10% w/v WPI. This example is similar to Example 2, above, but illustrates an alternative chemical modification of the alginate that results in a functionally similar crosslinked modified polysaccharide matrix embedded with milk proteins via physical chain entanglement.

Alg-GM was synthesized via aqueous reaction in the presence of glycidyl methacrylate. 3 g of alginate was dissolved in 250 ml of DI water, and flushed with nitrogen for 10 minutes. 18.4 g glycidyl methacrylate was added to the solution, flushed with nitrogen, and placed in an oil bath with condenser setup. The solution was stirred overnight at 60 C. The reactant solution was precipitated in cold ethanol, dried under vacuum, purified via dialysis in DI water, and lyophilized until dry. An Alg-GM/WPI solution was prepared by dissolving Alg-GM and WPI in DI water to final concentrations of 3% w/v and 10% w/v respectively. Alg-GM can be dissolved into a WPI solution, or vice versa. To enable photocrosslinking, a photoinitiator solution capable of releasing free radicals upon exposure to specific light wavelengths was added to the Alg-GM/WPI solution. This example utilized an Eosin Y/1-vinyl-2-pyrrolidone & triethanolamine system (5 μL and 50 μL in 2 mL polymer solution, respectively), which reacts when exposed to green light (525 nm). The hydrogel precursor solution was cast into a mold and crosslinked via exposure to 10 minutes of green light, after which a hydrogel was observed.

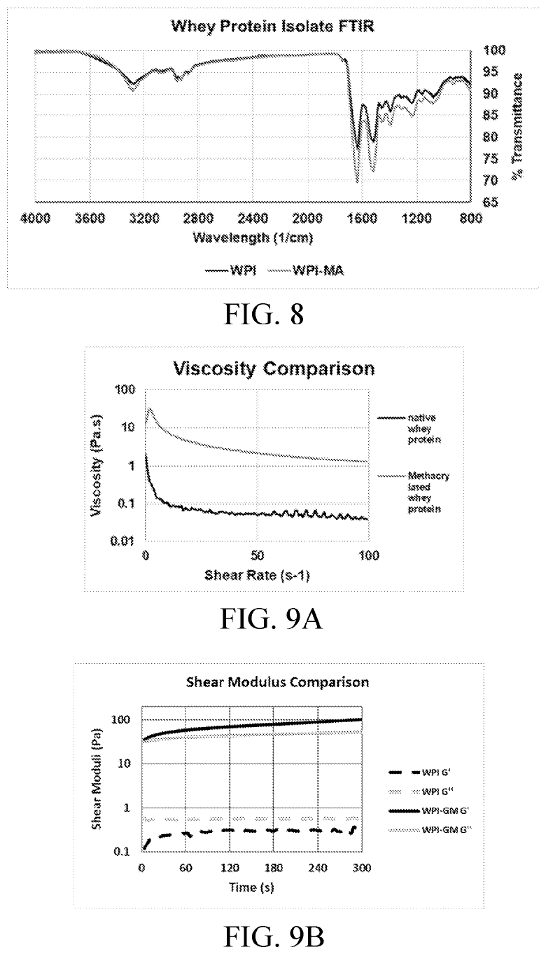

To help further verify modification of the whey protein of Examples 1 and 3, above, preliminary rheological studies were performed to assess the viscosity and shear moduli of the modified and non-modified materials. As evidenced in the plots of FIGS. 9A and 9B, there were distinct differences between the two materials. The viscosity of the modified material (FIG. 9A) was more than ten times greater than the viscosity of the non-modified protein. When looking at the shear moduli (FIG. 9B), several differences were noted as well. The non-modified material had a loss modulus (grey) greater than its storage modulus (black), which indicates a viscous, fluid-like behavior. The modified protein had a higher storage modulus, indicating an elastic, semi-solid behavior. Like the viscosity results, the moduli values of the modified protein were significantly higher than those of the non-modified protein. These increased values indicate a structural difference between the two materials, with the modified protein showing signs of increased interaction and potential bonding between protein chains as well as signs of network formation does not present in the non-modified protein. Additionally, the modified protein shows increases in moduli upon exposure to the visible light crosslinking system, indicating the presence of methacrylate groups.

FIG. 10 illustrates the swell ratio and weight loss for each of an example Alg-MA/WPI, such as the Alg-MA/WPI of Example 2, above, and an example Alg-MA/WPI-MA hydrogel, such as the Alg-MA/WPI-MA of Examples 1 and 3, above. An Alg-MA control is also shown for comparison. Alginate and WPI-based scaffold samples were placed in buffer (pH 7.4) in a shaker incubator at 37° C. At 24-hour intervals, samples were removed from solution and lyophilized. Initial weights, wet and final dry weights, were used for swell ratio and weight-loss calculations. The hydrogels all reached their maximum swell ratio within 24 hours, as no changes were subsequently seen throughout the 7 days of data collection. The samples which contained WPI showed significantly lower swell ratios, with over a 50% reduction in the swelling behavior compared to Alg-MA, which was expected due to the more hydrophobic nature of WPI. The swell ratios for samples which contained WPI and WPI-MA were similar. The sample weight loss measurements were taken after 24 hours, and similar to the swell ratio results, the materials lost weight within the first 24 hours with no subsequent significant changes throughout a 7-day period. The Alg-MA sample alone lost 20%. Incorporating the nonmodified WPI led to over a doubling in the weight loss, owing to the release of non-conjugated WPI within the Alg-MA network. As indicated, the Alg-MA/WPI-MA group did not lose a significant amount of weight compared to the control due to the covalent conjugation of WPI to the alginate network. FIG. 11 illustrates the unconfined compressive modulus for each of the example Alg-MA/WPI of FIG. 10, such as the Alg-MA/WPI of Example 2, above, and the example Alg-MA/WPI-MA hydrogel of FIG. 10, such as the Alg-MA/WPI-MA of Example 1/3, above. An Alg-MA control is also shown for comparison. All the samples were evaluated in unconfined, uniaxial compression directly after fabrication. The axial elastic modulus (i.e., stiffness) was calculated from the linear slope at the beginning of the stress-strain curve. Materials with non-modified WPI showed a decreased modulus compared to Alg-MA, with a reduction of nearly 50%; the material softening was due to the non-modified WPI physically interfering with the Alg-MA network formation (FIG. 8). The WPI-MA formed a covalent bond with Alg-MA, and the WPI part of the network provided elasticity to the more rigid Alg-MA structure (which is supported by the viscosity measurements above).

FIG. 12 shows viable cells via fluorescent staining performed on three examples of live-cell-seeded cell scaffolds illustrating the greater proliferation of cells in the cell scaffold that comprised both alginate (i.e. polymer) and WPI (i.e., milk protein). The assays were performed after 24 hours of c2cl2 proliferation in each of a seeded Alg-MA/WPI cell scaffold of the present disclosure and Alg-MA/WPI-MA cell scaffold of the present disclosure, as well as an Alg-MA control for comparison. As can be seen by comparing the images for the control (Alg-MA) on the left-hand side of FIG. 12 with each of the assay sets for the alginate-and whey-containing cell scaffolds in the middle and on the right-hand side of FIG. 12, the cell scaffolds comprising hydrogel containing both alginate and milk protein demonstrated much greater cell proliferation than the control, with the Alg-MA/WPI-MA cell scaffold outperforming the Alg-MA/WPI cell scaffold. These results demonstrate the efficacy of cell scaffolds made in accordance with the present disclosure.

In this example, the formation of alginate and whey hydrogels depended on the chemical modification of alginate and whey with the methacrylate molecule, enabling the two polymers to covalently crosslink (i.e., permanently bond) to each other in a controllable reaction. The methacrylated alginate and methacrylated whey were mixed together and then the mixture was exposed to a green light light-emitting diode (LED) system to induce the covalent crosslinking of alginate and whey to themselves and to each other. Images of the formed hydrogel can be seen in U.S. Provisional Patent Application Ser. No. 63/333,819, filed Apr. 22, 2022, and titled “Milk-Protein-Conjugated Biopolymers For In Vitro Meat Culture”, incorporated by reference herein.

Various modifications and additions can be made without departing from the spirit and scope of this disclosure. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present disclosure. Additionally, although particular methods herein may be illustrated and/or described as being performed in a specific order, the ordering is highly variable within ordinary skill to achieve aspects of the present disclosure. Accordingly, this description is meant to be taken only by way of example, and not to otherwise limit the scope of this disclosure. The appended claims as originally filed with this application shall be considered to form part of this Written Description section as if contained herein upon the filing of this application.

Exemplary embodiments have been disclosed above and illustrated in the accompanying drawings. It will be understood by those skilled in the art that various changes, omissions and additions may be made to that which is specifically disclosed herein without departing from the spirit and scope of the present disclosure.

1-109. (canceled)

110. A method of making a cultivated meat product having a desired three-dimensional (3D) shape, the method comprising:
providing a cell scaffold, wherein the cell scaffold:
includes an interconnecting network formed via crosslinking of a hydrogel in which plant-based polymer molecules and milk-protein molecules have been conjugated with one another; and
is formed into the 3D shape; and
seeding the interconnecting network with biological cells, wherein at least some of the biological cells are selected from a group consisting of myocytes and adipocytes.

111. The method of claim 110, wherein providing the cell scaffold includes chemically modifying the milk-protein molecules or the plant-based polymer molecules, or both the milk-protein molecules and the plant-based polymer molecules to enable crosslinking of the plant-based polymer molecules with one another or the milk-protein molecules with one another, or the plant-based polymer molecules with the milk-protein molecules or any combination thereof.

112. The method of claim 111, wherein providing the cell scaffold further includes mixing the milk-protein molecules and the polymer molecules with one another to create a mixture, wherein the milk-protein molecules conjugate upon mixing.

113. The method of claim 111, wherein providing the cell scaffold further includes:
mixing the milk-protein molecules and the plant-based polymer molecules with one another to create a mixture; and
after mixing, subjecting the mixture to an external stimulus so as to cause the plant-based polymer molecules and the milk-protein molecules to conjugate with one another.

114. The method of claim 110, wherein providing the cell scaffold includes chemically modifying the milk-protein molecules or the plant-based polymer molecules or both the milk-protein molecules and the plant-based polymer molecules via a crosslinking chemistry so as to enable conjugation of the plant-based polymer molecules with the milk-protein molecules.

115. The method of claim 114, wherein the milk-protein molecules are non-modified and are conjugated with modified ones of the plant-based polymer molecules via hydrophobic-hydrophobic interactions.

116. The method of claim 110, wherein providing the cell scaffold includes tuning the hydrogel to making the cultivated meat product, wherein the tuning includes A) tuning an amount of the plant-based polymer molecules, B) tuning an amount of the milk-protein molecules, C) tuning whether or how the plant-based polymer molecules are chemically modified, D) tuning whether or how the milk-protein molecules are functionalized, E) tuning in a manner in which the plant-based polymer molecules and the milk-protein molecules are conjugated with one another, or F) tuning a degree of conjugation between the plant-based polymer molecules and the milk-protein molecules, or any combination or sub-combination of A through F.

117. The method of claim 110, wherein providing the cell scaffold includes forming the hydrogel into the desired 3D shape by:
placing a hydrogel precursor solution to the hydrogel into a mold having a shape that matches the desired shape of the cell scaffold; and
causing the milk-protein molecules and the polymer molecules to conjugate with one another prior to removing the hydrogel from the mold.

118. The method of claim 110, wherein providing the cell scaffold includes forming the hydrogel into the desired 3D shape using the hydrogel as a bio-ink to print the cell scaffold.

119. The method of claim 110, wherein the cell scaffold is a porous 3D cell scaffold, and the method further comprises freeze-drying the hydrogel so as to form the porous 3D cell scaffold.

120. The method of claim 110, wherein providing the cell scaffold includes forming the hydrogel into the 3D shape:
mixing the hydrogel with a porogen; and
removing the porogen so as to form voids within the hydrogel.

121. The method of claim 110, wherein providing the cell scaffold includes forming the hydrogel into the 3D shape by building the cell scaffold using the hydrogel in a fibrous form.

122. The method of claim 110, wherein providing the cell scaffold includes forming the hydrogel into the 3D shape by building the cell scaffold using microcarrier particles of the hydrogel.

123. The method of claim 110, wherein the milk-protein molecules are recombinant milk-protein molecules.

124. The method of claim 110, wherein the milk-protein molecules are selected from the group consisting of whey protein isolate molecules, whey protein concentrate molecules, casein molecules, and beta-lactoglobulin molecules.

125. The method of claim 110, wherein the milk-protein molecules comprise or are a derivative of whey protein molecules.

126. The method of claim 110, wherein each of the milk-protein molecules is chemically modified to include a cell-adhesion functional group.

127. The method of claim 110, further comprising mixing the biological cells with a hydrogel precursor solution prior to conjugating the hydrogel, wherein the hydrogel is in a form of a hydrogel precursor solution prior to the conjugating, and the seeding of the interconnecting network includes seeding the biological cells at a density of about 10⁵ to about 10⁹ cells per milliliter of the hydrogel precursor solution.

128. The method of claim 110, wherein the seeding of the interconnecting network includes seeding the biological cells into the interstitial voids after the crosslinking of the hydrogel.

129. The method of claim 110, wherein at least some of the biological cells are selected from at least one of a satellite cell line, a primary cell line, and a non-differentiated cell line.

130. The method of claim 110, wherein at least some of the biological cells are selected from the group consisting of non-primate mammalian cells, reptilian cells, amphibian cells, and piscine cells, and any combination thereof.
